(19) United States
(12) Patent Application Publication
(10) Pub. No.: US 2005/0106712 A1
(43) Pub. Date: May 19, 2005

(54) Yellow low fluorescence dye for coated optical bead random array DNA analysis

(75) Inventors: Krishnan Chari, Fairport, NY (US); Tiecheng A. Qiao, Webster, NY (US); Donald R. Diehl, Rochester, NY (US); Samuel Chen, Penfield, NY (US)

Correspondence Address: Paul A. Leipold;Eastman Kodak Company, Patent Legal Staff, 343 State Street, Rochester, NY, 14650-2201, US

(73) Assignee: Eastman Kodak Company

(21) Appl. No.: 10/713,246

(22) Filed: Nov. 14, 2003

(51) Int. Cl. C12M 1/34; C07C 251/54

(52) U.S. Cl. 435/287.2; 564/147; 546/315; 548/537

(57) ABSTRACT

A coating composition for making a protein microarray, the composition comprising a gelling agent or a precursor to a gelling agent and microspheres; the microspheres containing a dye represented by Formula (I): 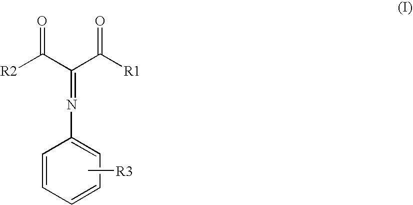 wherein: R1 and R2 independently reprresent substituted or unsubstituted alkyl, aryl, carbocyclic ring, heterocyclic ring, or amino; and R3 represents H, alkylamino, dialkylamino, hydroxy, or alkoxy.

### CROSS-REFERENCE TO RELATED APPLICATIONS

Reference is made to commonly-assigned, copending U.S. patent application Ser. No. ______ (DN85501), entitled MAGENTA LOW FLUORESCENCE DYE FOR COATED OPTICAL BEAD RANDOM ARRAY DNA ANALYSIS and copending U.S. patent application Ser. No. ______ (DN85500), entitled CYAN LOW FLUORESCENCE DYE FOR COATED OPTICAL BEAD RANDOM ARRAY DNA ANALYSIS, both filed of even date herewith. Reference is also made to U.S. Publication No. 2000-68609, filed Aug. 29, 2001, entitled RANDOM ARRAY OF MICROSPHERES. The copending applications are incorporated by reference herein for all that they contain.

### FIELD OF THE INVENTION

The present invention concerns biological microarray technology in general. In particular, the invention concerns the coloration of polystyrene microspheres or “beads” coated on a substrate to form a microarray.

### BACKGROUND OF THE INVENTION

Ever since it was invented in the early 1990s (Science, 251, 767-773, 1991), high-density arrays formed by spatially addressable synthesis of bioactive probes on a 2-dimensional solid support has greatly enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. Nos. 5,412,087, and 5,489,678 demonstrate the use of a photolithographic process for making peptide and DNA microarrays. The patent teaches the use of photolabile protecting groups to prepare peptide and DNA microarrays through successive cycles of deprotecting a defined spot on a 1 cm×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive. An ink jet approach is being used by others (e.g., U.S. Pat. Nos. 6,079,283; 6,083,762; and 6,094,966) to fabricate spatially addressable arrays, but this technique also suffers from high manufacturing cost in addition to the relatively large spot size of 40 to 100 μm.

Because the number of bioactive probes to be placed on a single chip usually runs anywhere from 1,000 to 100,000 probes, the spatial addressing method is intrinsically expensive regardless how the chip is manufactured. An alternative approach to the spatially addressable method is the concept of using fluorescent dye-incorporated polymeric beads to produce biological multiplexed arrays. U.S. Pat. No. 5,981,180 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information. U.S. Pat. No. 6,023,540 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach significantly expands the spectral barcoding capacity in microspheres. Further, it is reported in WO 02/077291 (Oct. 3, 2002) that beads used in the manufacture of microarrays may be colored with materials described as fluorophores or chromophores, in which WO 02/077291 defines fluorophore as a material which absorbs light and later emits light of a different wavelength, and chromophores as those materials which absorb light and do not emit light but instead convert the light into heat. It is reported in WO 02/077291 that the dyes imbibed in microspheres may be of the chromophore type but are preferred to be of the fluorophore type for the application described in WO 02/077291. Indeed, WO 02/077291 reports only the use of fluorophores in the examples described within the International Publication.

Even though the “spectrally addressed microsphere” approach does provide an advantage in terms of its simplicity over the old fashioned “spatially addressable” approach in microarray making, there are still needs in the art to make the manufacture of biological microarrays less difficult, less expensive, and to provide better dyes for coloration of microspheres.

### SUMMARY OF THE INVENTION

The present invention provides a dye for coloring microspheres yellow, i.e.—blue light absorbing, with colorant materials that have the property of very low fluorescence intensity such that the resultant colored microspheres do not substantially fluoresce when excited by visible light.

The present invention also provides a coating composition for making a protein microarray, the composition comprising a gelling agent or a precursor to a gelling agent and microspheres; the microspheres containing a dye represented by Formula (I): 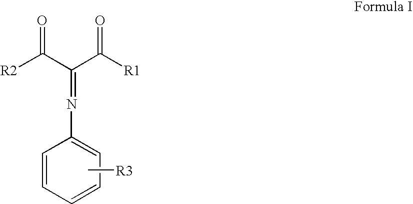
wherein: R1 or R2=substituted or unsubstituted alkyl, aryl, carbocyclic ring, heterocyclic ring, or amino; and, R3=H, alkylamino, dialkylamino, hydroxy, or alkoxy.

The invention utilizes a unique coating composition and technology to prepare a microarray on a substrate that may or may not be pre-etched with microwells and need not be premarked in any way with sites to attract the colored microspheres, as disclosed in the art. By providing the option of using unmarked substrates or substrates that need no pre-coating preparation, the present invention provides a huge manufacturing advantage compared to the existing technologies that do not provide the option. The invention discloses a method whereby color addressable mixed beads in a unique composition can be randomly distributed on a substrate that has no wells or sites to attract the microspheres.

The present invention provides a microarray that is less costly and easier to prepare than those previously disclosed, and further can be used in a colored microarray device such as described herein wherein red light absorbance is desired to be maximized and fluorescence of the dye imbibed in the colored polystyrene microsphere bead is desired to be minimized.

### BRIEF DESCRIPTION OF THE DRAWINGS

FIG. 1 is a diagram of a coated microsphere array device containing yellow colored microspheres 1, magenta colored microspheres 2 and cyan colored microspheres 3.

FIG. 2 is a schematic of a microspectrometer and fluorescence detection system used to characterize the colorant dye loaded in the microspheres.

FIG. 3 is a graph showing a spectroscopic response of YD-1 Dye loaded in a microsphere.

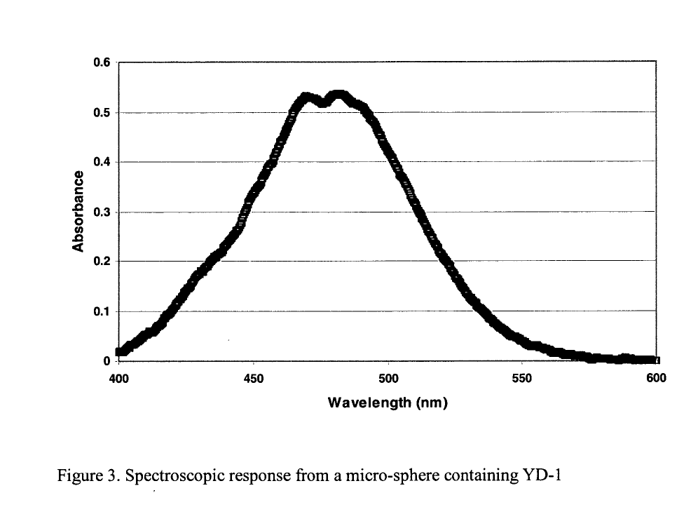

### DETAILED DESCRIPTION OF THE INVENTION

The coloration of a polystyrene microsphere beads requires that colorant materials are soluble in an organic solvent mixture that is designed to swell, but not dissolve, the polystyrene microsphere bead. Further, the colorant material must migrate from the solvent mixture into the polystyrene bead rendering the bead with high coloration. Further, the colorant must remain in the microsphere bead during the process of bead filtration, solvent removal and subsequent washing and de-swelling of the polystyrene beads. Organic colorant materials with high solubility in a mixture of acetone and toluene have been found to be most suitable to meet these requirements. In order for the dyes to impart strong coloration to the polystyrene microsphere beads it is also desirable that the colorant materials possess a high extinction coefficient. For the application described herein it is further desirable that the colorant material possess the property of no detectable fluorescence upon light exposure when imbibed in a polystyrene matrix. It is further desirable that the colorant materials possess such properties which impart a non-fading color to the polystyrene beads, and that the dye materials be easy to synthesize, and of low cost.

Those skilled in the art of colorant technology will recognize the difficulty of meeting all the requirements stated above. There are many classes of dyes and pigments known to the colorant art. Solubility in water, or organic solvents, has been a topic of high interest in the past for a wide variety of colorant applications, thus mathematical parameters have been devised to help the colorant scientist understand and predict dye or pigment solubility. However, there are no general guideline parameters with which a colorant scientist may predict the fluorescence of any given colorant material. Therefore, the colorant scientist must undertake an empirical approach to the discovery of colorant materials that are non-fluorescent. We have found that dye materials containing a specific halogen functionality are particularly likely to possess the property of very low fluorescence. Thus, the dyes of this invention have been found to have good solubility in the organic solvents required for bead coloration, high extinction, and remarkably low fluorescence when imbibed in a polystyrene microsphere bead.

The dyes that meet the above requirements for coloring polystyrene microsphere beads yellow, have been and found to posses the property of very low fluorescence, are described by the general formula I below and more preferably by the general formula II below: 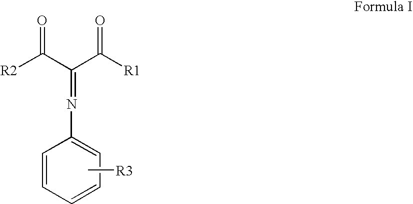
wherein: R1 and R2 independently can be substituted or unsubstituted alkyl, aryl, carbocyclic ring, heterocyclic ring, or amino; and R3 can be H, alkylamino, dialkylamino, hydroxy, or alkoxy; or preferably, Formula (II): 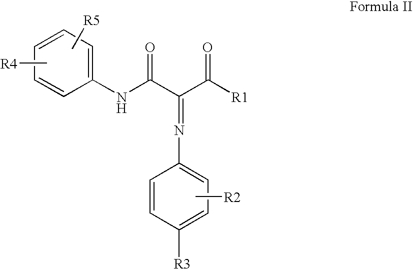
wherein:
R1 independently can be substituted or unsubstituted alkyl, aryl, carbocyclic ring, heterocyclic ring, or amino;
R2 can be one or more substituents selected from the group of H, alkyl, aryl, or heteroaryl;
R3 can be alkylamino, dialkylamino, or hydroxy;
R4 can be one or more substituents selected from the group of alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5 must be one or more substituents selected from the halogen group of Cl, Br, or I.

Dyes of the type described above are well known to those skilled in the art of photographic science. These dyes, known as azamethine (a.ka. azomethine) dyes, are typically formed in a photographic film or paper during processing of a color photographic material through aqueous processing of the photographic material in a processing bath containing an aryl amine known as a developer. We have found that these dyes surprisingly possess the quality of good solubility in solvents suitable for coloring polystyrene beads, and further specifically those dyes which possess halogen substitution (R5 above) on the arylamido portion of the dye chromophore have the very desirable property of extremely low fluorescence when imbibed into the polystyrene microsphere beads.

Examples of dyes that fulfill the requirements set out above are presented below, but one skilled in the art will recognize that the present invention is not limited thereto. Synthesis of dyes of this invention may be achieved by methods well known in the dye art as described in, for example: JP60032851, EP495406, JP01186952, U.S. Pat. No. 4,880,432, JP03079672, JP02292371, EP423796, U.S. Pat. No. 5,238,903, JP04126772, JP04239061, JP04252271, JP05069681, EP989452, WO9403835, WO0272721, and U.S. Pat. No. 6,489,511.

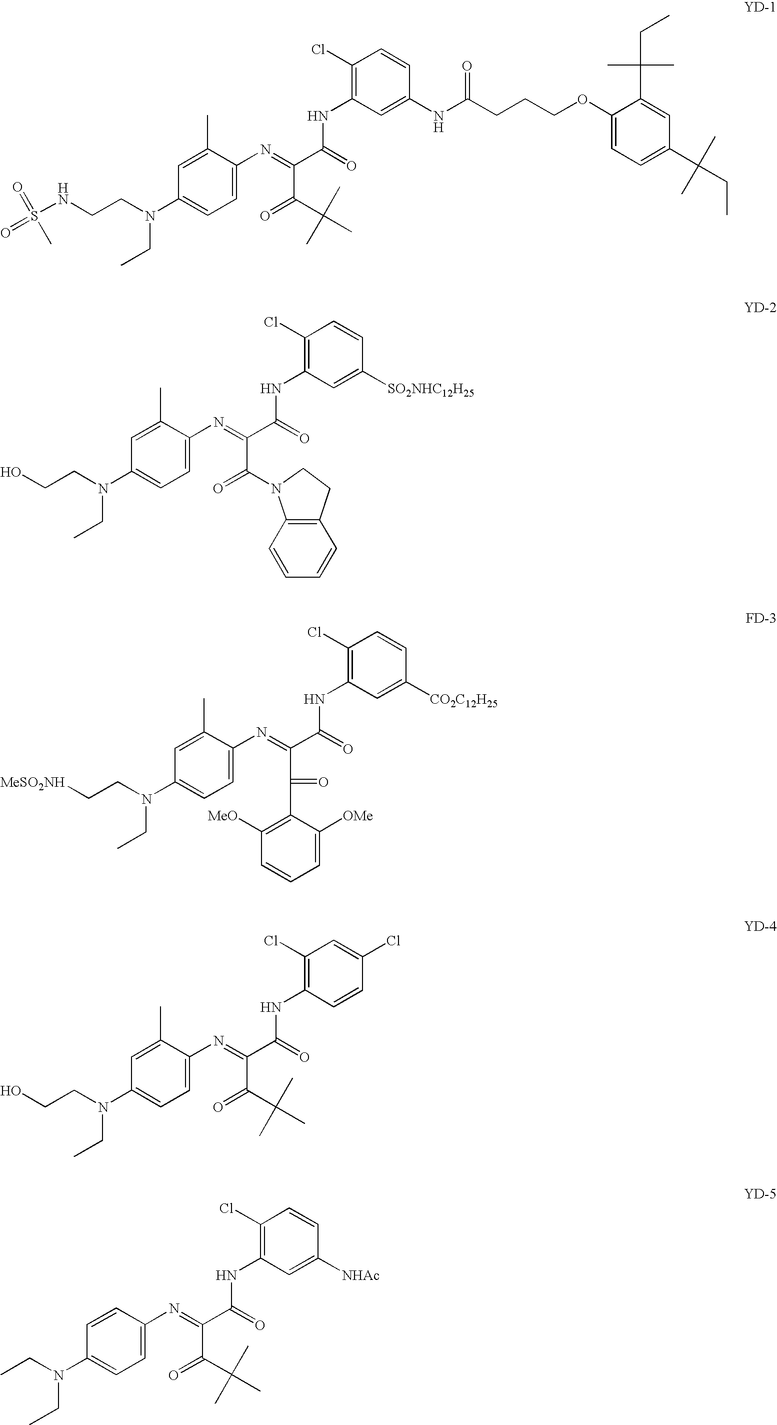

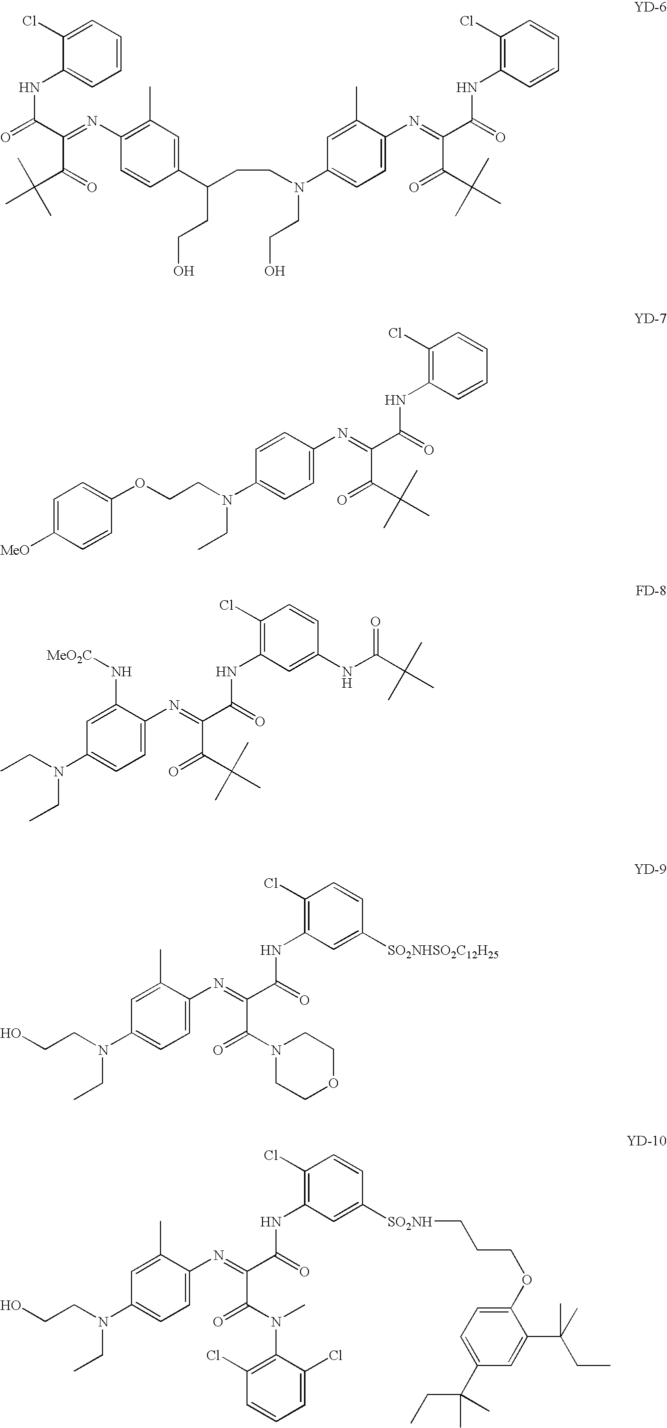

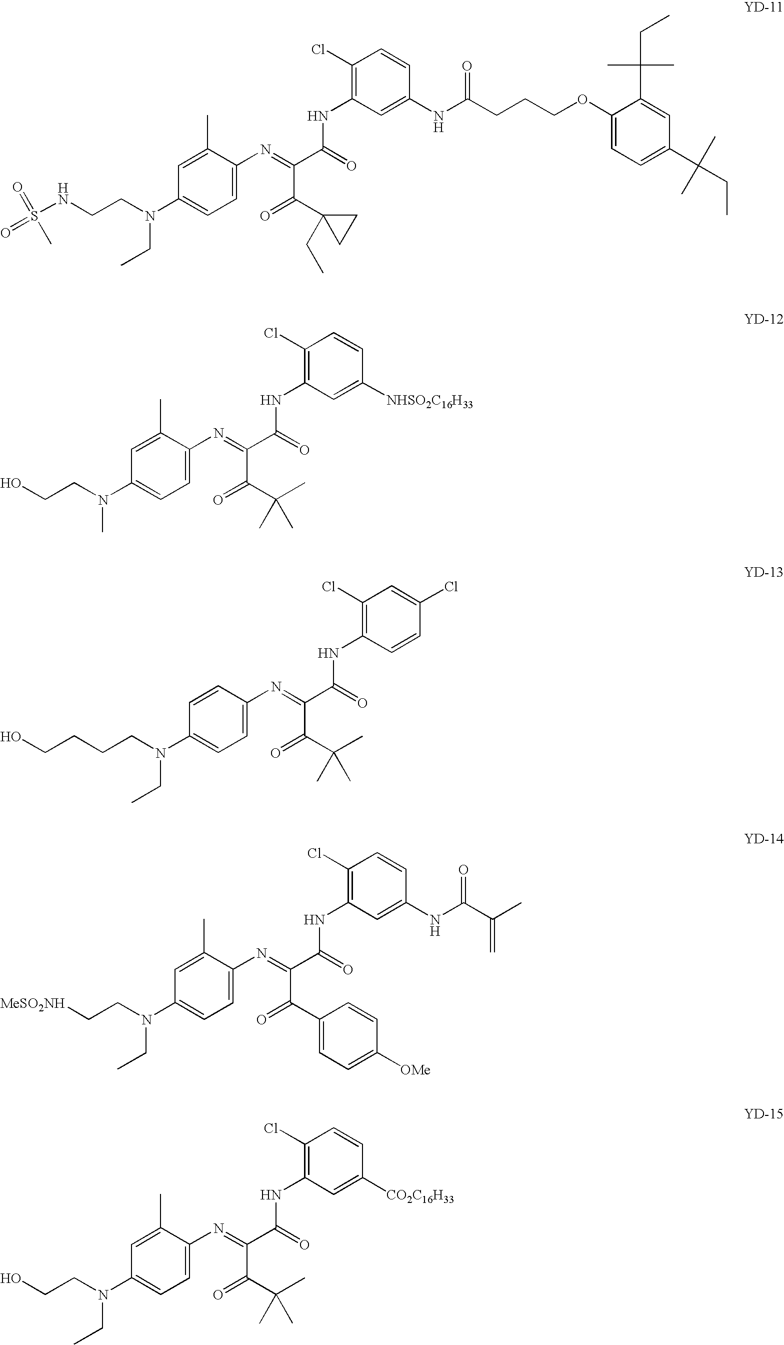

As used herein, the term “sol-to-gel transition” or “gelation” means a process by which fluid solutions or suspensions of particles form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation that is of the latter type. The process of gelation or setting is characterized by a discontinuous rise in viscosity. (See, P. I. Rose, “The Theory of the Photographic Process”, 4^{th} Edition, T. H. James ed. pages 51 to 67).

As used herein, the term “gelling agent” means a substance that can undergo gelation as described above. Examples include materials such as gelatin, water-soluble cellulose ethers or poly(n-isopropylacrylamide) that undergo thermal gelation or substances such as poly(vinyl alcohol) that may be chemically cross-linked by a borate compound. Other gelling agents may be polymers that may be cross-linked by radiation such as ultraviolet radiation. Examples of gelling agents include acacia, alginic acid, bentonite, carbomer, carboxymethylcellulose sodium, cetostearyl alcohol, colloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alcohol, povidone, propylene glycol alginate, sodium alginate, sodium starch glycolate, starch, tragacanth and xanthum gum. (For further discussion on gelling agents, see, accompanying reference Secundum Artem, Vol. 4, No. 5, Lloyd V. Allen). A preferred gelling agent is alkali pretreated gelatin.

As used herein, the term “random distribution” means a spatial distribution of elements showing no preference or bias. Randomness can be measured in terms of compliance with that which is expected by a Poisson distribution.

The present invention teaches a composition and a method for making a random array of colored microspheres, also referred to as “colored beads”, on a substrate. The distribution or pattern of the colored microspheres on the substrate is entirely random and the colored microspheres are not attracted or held to sites that are pre-marked or predetermined on the substrate as in other methods previously disclosed. In the present invention, the colored microspheres are immobilized randomly when the gelling agent in which they are carried undergoes a sol-to-gel transition.

The invention discloses a polymeric latex bead based random microarray with each colored bead in the array having a distinct signature that would distinguish the colored bead. Such a signature may be based on color, shape or size of the bead. For signatures based on color, the color may be derived from mixing three dyes representing the visual colors yellow, magenta, and cyan to create thousands of distinguishable beads with distinct “color addresses” (unique RGB values, e.g. R=0, G=204, B=153). The beads can be made with sites on their surface that are “active”, meaning that at such sites physical or chemical interaction can occur between the colored bead and other molecules or compounds. Such compounds may be organic or inorganic. Usually, the molecule or compound is organic—nucleic acid, protein or fragments thereof, are examples. To the surface of each color coded bead may be attached a pre-synthesized oligonucleotide, a monoclonal antibody, or any other biological agents. Therefore, each color address can correspond to a specific bioactive probe. These colored beads may be mixed in equal amounts, and the random microarray fabricated by coating the mixed beads in a single or multilayer format.

Coating methods are broadly described by Edward Cohen and Edgar B. Gutoff in Chapter 1 of “Modern Coating And Drying Technology”, (Interfacial Engineering Series; v.1), (1992), VCH Publishers Inc., New York, N.Y. For a single layer format, suitable coating methods may include dip coating, rod coating, knife coating, blade coating, air knife coating, gravure coating, forward and reverse roll coating, and slot and extrusion coating.

Fluorescently/chemiluminescently labeled biological sample can be hybridized to the bead based random microarray. The signals from both “color addressable” polymeric beads and biological sample non-selectively labeled with fluorescence/chemiluminescence may be analyzed by a charge coupled device after image enlargement through an optical system. The recorded array image can be automatically analyzed by an image processing algorithm to obtain bioactive probe information based on the RGB color code of each bead, and the information compared to the fluorescence/chemiluminescence image to detect and quantify specific biological analyte materials in the sample. Optical or other electro-magnetic means may be applied to ascertain signature.

Although microspheres or particles having a substantially curvilinear shape are preferred because of ease of preparation, particles of other shape such as ellipsoidal or cubic particles may also be employed. Suitable methods for preparing the particles are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used. The formed microsphere is colored using an insoluble colorant that is a pigment or dye that is not dissolved during coating or subsequent treatment. Suitable dyes may be oil-soluble in nature. It is preferred that the dyes are non-fluorescent when incorporated in the microspheres.

The microspheres are desirably formed to have a mean diameter in the range of 1 to 50 microns; more preferably in the range of 3 to 30 microns and most preferably in the range of 5 to 20 microns. It is preferred that the concentration of microspheres in the coating is in the range of 100 to a million per cm², more preferably 1000 to 200,000 per cm² and most preferably 10,000 to 100,000 per cm².

The attachment of bioactive agents to the surface of chemically functionalized microspheres can be performed according to the published procedures in the art (Bangs Laboratories, Inc, Technote #205). Some commonly used chemical functional groups include, but not limited to, carboxyl, amino, hydroxyl, hydrazide, amide, chloromethyl, epoxy, aldehyde, etc. Examples of bioactive agents include, but are not limited to, oligonucleotides, DNA and DNA fragments, PNAs, peptides, antibodies, enzymes, proteins, and synthetic molecules having biological activities.

### EXAMPLES

### Example 1

This example illustrates the colorant density obtained by using dye YD-1 (whose synthesis is described in JP 60032851) to color polystyrene microsphere beads.

A 4.2% aqueous suspension of polystyrene beads prepared by emulsion polymerization and having a mean size of 10 micrometers was obtained from Interfacial Dynamics Corporation, Portland, Oreg.

A suspension of yellow colored beads YD-1 was prepared by first dissolving 0.006 grams of YD-1 in 0.02 grams of toluene and 2 grams of acetone. 2.5 grams of the suspension of the above suspension of non-dyed (clear) beads was combined with 3 grams of acetone. This mixture was then added rapidly to the solution of YD-1 in acetone and toluene while stirring to prepare a suspension of colored beads. The suspension of colored beads was then filtered using a porous cotton filter, poured into a dialysis bag (12,000 to 14,000 molecular weight cut off) and washed with distilled water for one hour. fter washing, the suspension of colored beads was filtered again using a porous cotton filter.

Spectroscopic responses and fluorescence intensity levels in colorant-dyed microspheres were analyzed using a hybrid analytical system comprising three parts: optical microscope, fluorescence microscope, and ultra-violet visible (UV-VIS) micro-spectrometer. This system uses high-intensity light, lenses, mirrors, apertures and optical detectors to not only generate a magnified image of the coated array of microspheres, but also selectively capture the spectroscopic response of individual colored microspheres.

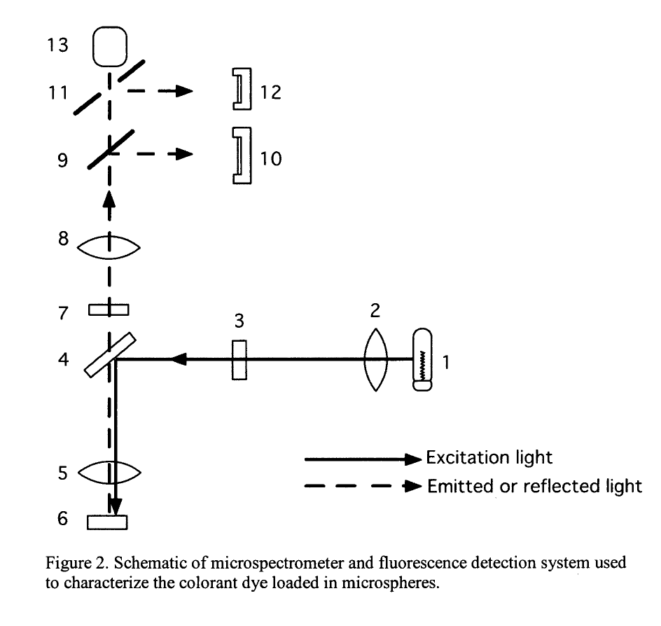

The spectroscopic response of individual bead is obtained in a microspectrometer set up, consisting of an optical microscope and a spectrophotometer. The procedure for obtaining this response starts by first obtaining a magnified image of the microspheres. This is performed by focusing light from a light source, 1 in FIG. 2, (e.g. halogen or xenon lamp), through the collector lens assembly, 2, down a dichroic mirror, 4, and onto the microarray specimen, 6. The reflected light is then focused by the objective lens, 6, so that a magnified image of a given field of view can be captured. The removable mirror, 9, controls the option of image capture by the digital camera, 10, or spectral capture by the micro-spectrometer, 13. The optical microscope used here is an Olympus BX-30MFSP modular optical system (from Olympus PID Corp, Woodbury, N.Y.), equipped with a Spot RT-Slider Camera (from Diagnostic Instruments, Inc.). Optical microscopy and fluorescence microscopy methods are broadly described by D. B. Murphy, “Fundamentals of Light Microscopy and Electronic Imaging”, Wiley-Liss, Inc. Publishing, 2001; and D. J. Goldstein, “Understanding the Light Microscope. A Computer-aided Introduction”, Academic Press, California, 1999. Depending on the magnification used, optical microscope imaging can provide the location of hundreds to thousands of beads in a single field of view. The combination of many images can provide the location to tens and hundreds of thousands of bead locations.

Once the locations of the microspheres are known, each bead can then be analyzed by spectroscopy by translating the bead of interest, as revealed in the optical microscope image obtained by the procedure described above, into a position for spectral capture. For color analysis of individual microspheres in a random array of mixed color microspheres, our use of UV-VIS spectrometry uniquely allows both the color type and colorant concentration to be obtained. This component is comprised of an F-40 light gathering optics setup (Filmetrics Inc., San Diego) that holds a 45° angled mirror etched with a small aperture, 11 in FIG. 2. This feature permits the extraction of spectral information from a specific region, even from a select region within a 10 micron diameter bead. The spectral information is then collected on the spectrometer sensor, 13 (USB-2000, OceanOptics, FL), and processed with the OOIBase32 software (from OceanOptics, FL).

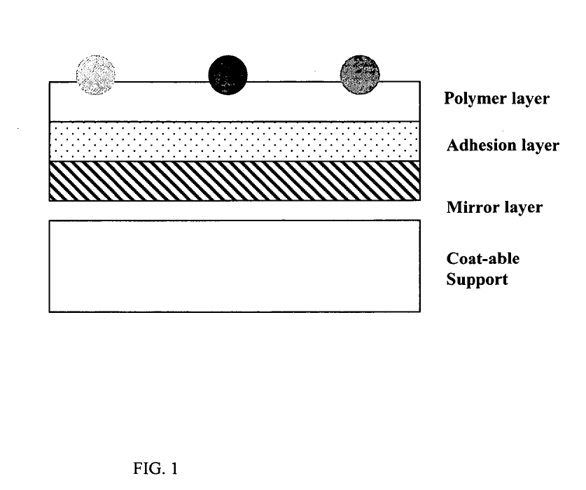

Two-dimensional translation of the substrate, containing the micro-array, in FIG. 1, allows a bead of interest to be positioned within the spectrometer aperture, 11 of FIG. 2. Changes in the magnifying power of the objective lens, 5 of FIG. 2, and the variable zoom lens, 8, allows different amount of the bead area to be confined by the aperture opening. For analysis of colors in these microspheres, it is preferred that at least two times the area defined by the diameter, D, of the bead is within the aperture opening, i.e. an area of the squared length dimension, π(D/2)², containing the bead of interest. More preferably, one time the area based on the diameter of microsperhere is used, and most preferably, 0.5 times the diameter region, in the central portion of the bead, is selected by the aperture opening.

To obtain the color type and color level of colorant in the microsphere, the spectral intensity response in the 300-1000 nm wavelength region of the electromagnetic radiation spectrum is collected, and processed (e.g. as Absorbance, A) following the relationship:

$$Absorbance , A = - log \frac{( {I}_{reference} - {I}_{background} )}{( {I}_{sample} - {I}_{background} )}$$

where
I_{reference}=intensity response of a bead without colorant,
I_{background}=null intensity with zero incident light
Iₛₐₘₚₗₑ=intensity response of colored bead

It is known in the field that the absorbance, A, is related to the concentration of the light absorbing specie in the microsphere by Beer's Law:
A=εbc, where
ε is the molar absorptivity of the colorant in the bead,
b is the path length of the microsphere traversed by the light
c is the concentration of the colorant in the bead.
Therefore,

$$- log \frac{( {I}_{reference} - {I}_{background} )}{( {I}_{sample} - {I}_{background} )} = {ɛ}_{.} bc$$

Therefore, the measured intensity ratio is a monitor of the colorant concentration. The theory and practice of UV-VIS spectroscopy as followed in this disclosure is broadly described by D. A. Skoog and J. J. Leary, in the book “Principles of Analytical Chemistry”, Chapter 6 and 7, Saunders College Publishing, 1992. The processed data can be displayed, for example as Absorbance vs. Wavelength plots, so that changes in colorant spectral response (for identifying its color type), and intensity levels (for identifying color concentration levels) can be collected and evaluated.

For the invention dye described in this application, the measured spectral characteristics showed a wavelength response with a peak at about 470 nm, and a full width at half maximum in the range 440-510 nm. This intensity variation in spectral response over the 400-600 nm range indicates that the colorant in the bead absorbs primarily in the blue region of the visible light spectrum.

As shown, the yellow dyes of the present invention are excellent at coloring polystyrene micro-sphere beads.

### Example 2

This example illustrates the very low fluorescence obtained in polystyrene microsphere beads colored with YD-1.

Comparison of the fluorescence intensity variation in several yellow dyes was made using the hybrid analytical system described above. By incorporating fluorescence cubes, each consisting of an exciter filter, 3 in FIG. 2, a dichroic mirror, 4, and a barrier filter, 7, the fluorescence emission characteristics of a colorant can be monitored. The spectroscopy data was used to select the wavelength characteristics of the fluorescence cube. For the yellow dyes used in this invention application, the excitation filter was selected to band pass light in the 550-580 nm region, the dichroic mirror was capable of passing light >600 nm in wavelength, and the barrier filter had a high pass characteristic of >610 nm. By setting the spectrometer to collect fluorescence emission intensity for a fixed time (1 second), while keeping other experimental conditions constant, comparison of the fluorescence intensity between different dyes were made, Table 1.

TABLE 1
Fluorescence Emission of Colored Beads

| | Maximum Intensity of |
|---|---|
| | Fluorescence Emission |
| Dye | (arbitrary units) |
| YD-1 | less than 10 |
| Neopen Yellow 075 (BASF Corp.) | 40 |
| Sudan Orange 220 (BASF Corp.) | 300 |
| Sandoplast Yellow 3G (Clariant Corp.) | 240 |

As shown here, the polystyrene micro-spheres have the least fluorescence when imbibed with YD-1.

1. A coating composition for making a protein microarray, the composition comprising a gelling agent or a precursor to a gelling agent and microspheres; the microspheres containing a dye represented by Formula (I): 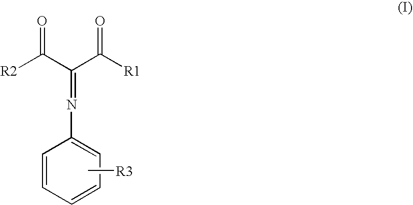
wherein: R1 and R2 independently reprresent substituted or unsubstituted alkyl, aryl, carbocyclic ring, heterocyclic ring, or amino; and R3 represents H, alkylamino, dialkylamino, hydroxy, or alkoxy.

2. The coating composition according to claim 1 wherein the microspheres contain a dye represented by Formula (II): 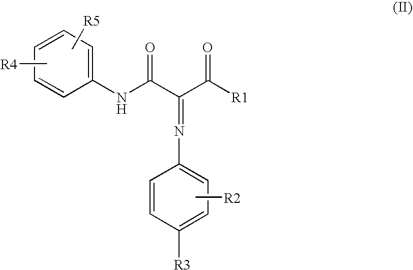
wherein:
R1 is substituted or unsubstituted alkyl, aryl, carbocyclic ring, heterocyclic ring, or amino;
R2 is one or more substituents selected from H, alkyl, aryl, or heteroaryl;
R3 is alkylamino, dialkylamino, or hydroxy;
R4 is one or more substituents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5 must be one or more substituents selected from the halogen group of Cl, Br, or I.

3. The coating composition according to claim 2 wherein:
R1=substituted or unsubstituted alkyl;
R2=H, or substituted or unsubstituted alkyl;
R3=Disubstituted amino;
R4=one or more substitutents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5=one or more Cl.

4. The coating composition according to claim 1 wherein the gelling agent is gelatin.

5. The coating composition according to claim 1 wherein the gelling agent undergoes thermal gelation.

6. The coating composition according to claim 4 wherein the gelatin is alkali pretreated gelatin.

7. The coating composition according to claim 1 wherein the microspheres have a mean diameter between 1 and 50 microns.

8. The coating composition according to claim 1 wherein the microspheres have a mean diameter between 3 and 30 microns.

9. The coating composition according to claim 1 wherein the microspheres have a mean diameter between 5 and 20 microns.

10. The coating composition according to claim 1 wherein the microspheres comprise a synthetic or natural polymeric material.

11. The coating composition according to claim 10 wherein the polymeric material is an amorphous polymer.

12. The coating composition according to claim 11 wherein the amorphous polymer is polystyrene.

13. A microarray comprising:
a substrate coated with a composition comprising
a gelling agent or a precursor to a gelling agent and microspheres; the microspheres containing a dye represented by Formula (I): 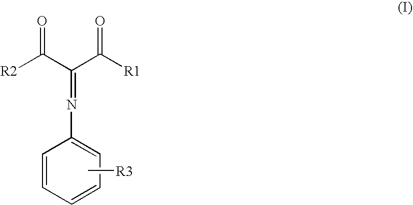
wherein: R1 and R2 independently represent substituted or unsubstituted alkyl, aryl, carbocyclic ring, heterocyclic ring, or amino; and R3 represents H, alkylamino, dialkylamino, hydroxy, or alkoxy;
wherein the microspheres are immobilized on the substrate.

14. The microarray according to claim 13 wherein the microspheres contain a dye represented by Formula (II): 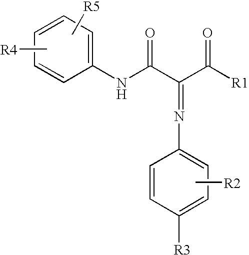
wherein:
R1 is substituted or unsubstituted alkyl, aryl, carbocyclic ring, heterocyclic ring, or amino;
R2 is one or more substituents selected from H, alkyl, aryl, or heteroaryl;
R3 is alkylamino, dialkylamino, or hydroxy;
R4 is one or more substituents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5 must be one or more substituents selected from the halogen group of Cl, Br, or I.

15. The microarray according to claim 14 wherein:
R1=substituted or unsubstituted alkyl;
R2=H, or substituted or unsubstituted alkyl;
R3=Disubstituted amino;
R4=one or more substitutents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5=one or more Cl.

16. The microarray according to claim 13 wherein the gelling agent is gelatin.

17. The microarray according to claim 13 wherein the substrate comprises glass, plastic, cellulose acetate, or polyethyleneterephthalate.

18. The microarray according to claim 13 wherein the substrate is flexible.

19. The microarray according to claim 13 wherein the microspheres are immobilized on the substrate in a concentration between 100 and 1 million microspheres per cm².

20. The microarray according to claim 13 wherein the microspheres are immobilized on the substrate in a concentration between 1000 and 200,000 microspheres per cm².

21. The microarray according to claim 13 wherein the microspheres are immobilized on the substrate in a concentration between 10,000 and 100,000 microspheres per cm².

22. The microarray according to claim 13 wherein the microspheres are immobilized on the substrate upon gelation of the gelling agent.

23. The microarray according to claim 13 wherein the microspheres carry surface active sites.

24. The microarray according to claim 23 wherein the surface active sites carry organic or inorganic attachments.

25. The coating composition according to claim 23 wherein the surface active sites are capable of chemical or physical interaction.

26. The microarray according to claim 23 wherein the surface active sites are bioactive.

27. The microarray according to claim 26 wherein the bioactive sites interact with nucleic acid, protein, or fragments thereof.

28. The microarray of claim 13 wherein the microspheres are randomly distributed on the substrate.

29. The microarray of claim 13 wherein the substrate is characterized by an absence of specific sites capable of interacting physically or chemically with the microspheres.

30. A method of making a microarray comprising the steps of:
providing a substrate; and
coating on the substrate the composition according to claim 1;
wherein said composition is fluid during coating and the microspheres become immobilized in the plane of the coating due to sol-gel transition.

31. The method of claim 29 wherein the microspheres become randomly immobilized on the substrate.

32. A microsphere for making an array, the microsphere comprising a capsule containing a dye represented by Formula (I): 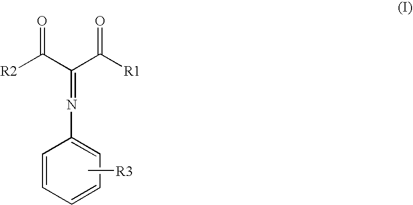
wherein: R1 and R2 independently reprresent substituted or unsubstituted alkyl, aryl, carbocyclic ring, heterocyclic ring, or amino; and R3 represents H, alkylamino, dialkylamino, hydroxy, or alkoxy.

33. A microsphere for making an array, the microsphere comprising a capsule containing a dye represented by Formula (II): 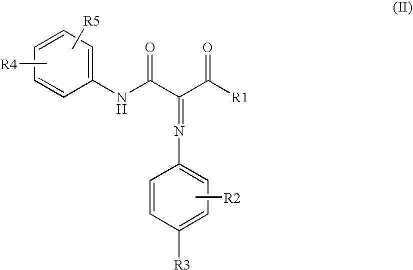
wherein:
R1 is substituted or unsubstituted alkyl, aryl, carbocyclic ring, heterocyclic ring, or amino;
R2 is one or more substituents selected from H, alkyl, aryl, or heteroaryl;
R3 is alkylamino, dialkylamino, or hydroxy;
R4 is one or more substituents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5 must be one or more substituents selected from the halogen group of Cl, Br, or I.

34. The microsphere according to claim 33 wherein:
R1=substituted or unsubstituted alkyl;
R2=H, or substituted or unsubstituted alkyl;
R3=Disubstituted amino;
R4=one or more substitutents selected from alkyl or substituted alkyl, acyl or substituted acyl, amido or substituted amido, substituted sulfonyl, substituted sulfamoyl, nitro, aryl or substituted aryl, or heteroaryl or substituted heteroaryl; and
R5 one or more Cl.
